(19) United States
(12) Patent Application Publication
(10) Pub. No.: US 2012/0078114 A1
(43) Pub. Date: Mar. 29, 2012

(54) System and method for real-time perfusion imaging

(75) Inventors: Steven H. Mersch, Germantown, OH (US); Jennifer J. Whitestone, Germantown, OH (US)

(73) Assignee: Point of Contact, LLC

(21) Appl. No.: 12/924,451

(22) Filed: Sep. 28, 2010

(51) Int. Cl. A61B 6/00

(52) U.S. Cl. 600/475; 600/477; 600/476

(57) ABSTRACT

The present invention relates to a system and method for real-time perfusion imaging. More particularly, the present invention relates to a perfusion imaging system including a plurality of coaligned imaging arrays operating under a specific timing sequence and method of using the same.

### BACKGROUND OF THE INVENTION

(a) Field of the Invention

The present invention relates to a system and method for real-time perfusion imaging. More particularly, the present invention relates to a perfusion imaging system including a plurality of coaligned imaging arrays operating under a specific timing sequence and method of using the same.

(b) Background of the Invention

Skin, the largest organ of the body, has been essentially ignored in medical imaging. No standard of care regarding skin imaging exists. Computerized Tomography (“CT”), Magnetic Resonance Imaging (“MRI”), and ultrasound are routinely used to image the body for signs of disease and injury. Researchers and commercial developers continue to advance these imaging technologies to produce improved pictures of internal organs and bony structures. Clinical use of these technologies to diagnose and monitor subsurface tissues is now a standard of care. However, no comparable standard of care exists for imaging skin. Skin assessment has historically relied on visual inspection augmented with digital photographs. This method does not take advantage of the remarkable advances in nontraditional surface imaging, and lacks the ability to quantify the skin's condition, prohibiting the clinician's ability to diagnose and monitor skin-related ailments. Electronically and quantitatively recording the skin's condition with different surface imaging techniques will aid in staging skin-related illnesses that affect a number of medical disciplines such as plastic surgery, wound healing, dermatology, endocrinology, oncology, and trauma.

Other skin injuries include trauma and burns. Management of patients with severe burns and other trauma is affected by the location, depth, and size of the areas burned, and also affects prediction of mortality, need for isolation, monitoring of clinical performance, comparison of treatments, clinical coding, insurance billing, and medicolegal issues. Current measurement techniques, however, are crude visual estimates for burn location, depth, and size. Depth of the burn in the case of an indeterminate burn is often a “wait and see” approach. Accurate initial determination of burn depth is difficult even for the experienced observer and nearly impossible for the occasional observer. Total Burn Surface Area (“TBSA”) measurements require human input of burn location, severity, extent, and arithmetical calculations, with the obvious risk of human error.

Perfusion imaging is a developing method for determining microcirculatory flow. Commercial laser Doppler scanners, one means of perfusion imaging, have been used in clinical applications that include determining burn injury, rheumatoid arthritis, and the health of post-operative flaps. During the inflammatory response to burn injury, there is an increase in perfusion. Laser Doppler Imaging (“LDI”), used to assess perfusion, can distinguish between superficial burns, areas of high perfusion, and deep burns, areas of very low perfusion. LDI has also been finding increasing utility in dermatology. LDI has been used to study allergic and irritant contact reactions, to quantify the vasoconstrictive effects of corticosteroids, and to objectively evaluate the severity of psoriasis by measuring the blood flow in psoriatic plaques. It has also been used to study the blood flow in pigmented skin lesions and basal cell carcinoma where it has demonstrated significant variations in the mean perfusion of each type of lesion, offering a noninvasive differential diagnosis of skin tumors.

When a diffuse surface such as human skin is illuminated with coherent laser light, a random light interference effect known as a speckle pattern is produced in the image of that surface. If there is movement in the imaging subject, such as capillary blood flow, the speckles fluctuate in intensity. The intensity fluctuation may be captured and recorded by an imaging device, which provides data regarding tissue perfusion. LDI techniques for blood flow measurements are based on this phenomenon.

A current approach involves illuminating a region of interest with coherent laser light and imaging the region of interest using an exposure time long enough to allow blurring of the speckle pattern where motion is occurring. The degree of blurring is correlated with perfusion of the imaging subject. While this method produces a perfusion image, the approach suffers reduced spatial resolution and blurring by artifacts. An alternative approach is to acquire a sequence of images using a very fast imaging array. Time-dependent fluctuations in the speckle pattern are used to measure perfusion under both techniques, whether by analyzing the blurring of the pattern under the first approach or analyzing the differences between sequential images under the second approach. While LDI is becoming a standard, it is limited by specular artifacts, low resolution, and long measurement times.

Imaging blood flow is an essential tool to assess many physiological processes and diseases. Several instruments to produce blood flow images have been proposed. However, these instruments include significant limitations imposed by signal sampling frequency requirements. The measured frequencies in laser Doppler blood flowmetry are typically in the range of 0 to 20 kHz. In order to address this frequency requirement, some instruments use a CMOS imaging array. CMOS image sensors allow for fast sub-frame rates as pixels can be addressed randomly. However, a limitation arises from the electronic architecture of a particular photosensor matrix. The main requirement is a need to selectively read out the pixels from a predefined sub-frame at high-speed. Ideally, the sub-frames would be acquired at up to 40,000 frames per second as assumed for the maximum sampling frequency in laser Doppler flowmetry. This is a very high frame rate even for sub-frames. Normal camera full frame rates are 30 or 60 frames per second. This need for an extremely high sub-frame rate places severe requirements on the image processing software, requires a powerful, expensive, and potentially dangerous light source to provide sufficient illumination, and limits the quality and resolution of the results.

### SUMMARY OF THE INVENTION

The present invention addresses the shortcomings of the prior art and provides an improved system and method for perfusion imaging. The present invention provides increased resolution, faster performance, lower power lighting requirements, and a lower cost than current technologies. More particularly, the present invention relates to a perfusion imaging system including a plurality of coaligned imaging arrays operating under a specific timing sequence and method of using the same.

The key innovation in the system and method described herein is the use of a plurality of complimentary metal oxide semiconductor (“CMOS”) imaging arrays co-aligned to the same field of view. A neutral separation prism directs identical images to preferably up to five identical CMOS imaging arrays. Each array uses an identical filter or uses no filter at all. Each imaging array includes controllable timing, such that each imaging array may sequentially acquire a temporally offset image. Comparing the differences in signal between temporally offset arrays on a pixel by pixel basis provides a speckle fluctuation history, indicating the microcirculatory flow of the imaging sample and allowing the creation of a perfusion image of the imaging sample.

In one embodiment, the present invention is an apparatus for imaging and measuring time-based fluctuations in a laser speckle pattern, comprising: a plurality of coaligned imaging arrays, each with independently controllable timing; a coherent light source arranged for illuminating at least a portion of an imaging subject; a lens for collecting light from the imaging subject; a prism for presenting identical spectral content from the lens to each of the plurality of imaging arrays; a central processing unit in electronic communication with the coherent light source and each of the plurality of imaging arrays; a control set in electronic communication with the central processing unit, the control set for controlling the coherent light source and the plurality of imaging arrays; and a display in electronic communication with the central processing unit. Preferably, this embodiment further comprises a spatial filter arranged for restricting the cone angle of light reaching the lens, wherein the spatial filter includes a plurality of microchannels, each microchannel including a diameter and a length. The plurality of microchannels are an array of microchannels arranged in a honeycomb pattern, and each of the plurality of microchannels is hexagonal in shape. Ideally, each of the plurality of imaging arrays comprises a two-dimensional array of pixels, and the spatial filter includes a plurality of microchannels with a diameter/length ratio selected to produce a speckle approximately equal in size to one of the pixels.

In this embodiment, each of the plurality of imaging arrays comprises a non-integrating two-dimensional array of pixels. Further, each of the plurality of imaging arrays sequentially acquires image data, and the sequentially acquired image data are separated by a user-determined temporal offset, about 50 μsec. In this embodiment, a perfusion image is created by calculating pixel intensity fluctuations between the sequentially acquired image data. Preferably, each of the plurality of imaging arrays acquires image data at a frame rate between about 30-60 frames per second.

In this embodiment, the coherent light source emits at a wavelength of at least 400 nm and at a power of less than 500 mW, and preferably, emits at a wavelength between about 630-850 nm and at a power of about 10 mW. The plurality of imaging arrays further comprising a band pass filter arranged to selectively pass light at about the wavelength of the coherent light source. The apparatus may be incorporated into a convergent parameter instrument. The independently controllable timing of each of the plurality of imaging arrays may be controlled by a user. The plurality of imaging arrays is preferably one of three, four, and five imaging arrays, and the display is preferably a touch screen display. The apparatus may further comprise a data transfer unit in electronic communication with the central processing unit and computer readable storage media in electronic communication with the central processing unit.

In a further embodiment, the present invention is a method of producing a perfusion image comprising the steps of: (a.) illuminating at least a portion of an imaging subject using a coherent light source; (b.) collecting spectral content from the imaging subject; (c.) directing the spectral content to each of a plurality of imaging arrays; (d.) sequentially acquiring an image with each of the plurality of imaging arrays; (e.) processing the sequentially acquired images to generate a single perfusion image; and (f.) displaying the perfusion image on a display. The sequentially acquired images of step (d.) are separated by a user-determined temporal offset, and each of the sequentially acquired images is represented as a two dimensional array of numerical values. The processing of the sequentially acquired images in step (e.) comprises: (a.) calculating an absolute value array for each pair of sequentially acquired images, whereby each position in the absolute value array is the absolute value of the difference in numerical values at corresponding positions in the temporally adjacent pair of sequentially acquired images; (b.) determining if only a single absolute value array was calculated in step (a.): (1.) if so, defining the single absolute value array as a final array and proceeding to step (c.); (2.) if not, calculating an additive array, whereby each position in the additive array is the sum of numerical values at corresponding positions in each of the absolute value arrays, defining the additive array as a final array, and proceeding to step (c.); and (c.) assigning a color to each position within the final array based on the numerical value at that position to generate a perfusion image.

In this embodiment, the method is performed by a central processing unit in electronic communication with the coherent light source and the plurality of imaging arrays. Further, steps (a.)-(f.) are repeated at a rate between about 30-60 Hz for displaying real-time perfusion imaging. Preferably, the imaging subject is an individual's skin.

### BRIEF DESCRIPTION OF THE DRAWINGS

A better understanding of the present invention will be had upon reference to the following description in conjunction with the accompanying drawings, wherein:

FIG. 1 is a block diagram of an embodiment of a perfusion imaging system

FIG. 2A depicts a two channel prism;

FIG. 2B depicts a three channel prism;

FIG. 2C depicts a four channel prism;

FIG. 2D depicts a five channel prism;

FIG. 3A is a flowchart of a method for real-time perfusion imaging;

FIG. 3B is an example of the method for real-time perfusion imaging;

FIG. 4A depicts a perspective view of a microchannel array spatial filter;

FIG. 4B depicts a detail top view of the microchannel array spatial filter; and

FIG. 5 is a schematic diagram depicting the effect of the microchannel array spatial filter on light rays.

### DETAILED DESCRIPTION OF THE PREFERRED EMBODIMENT

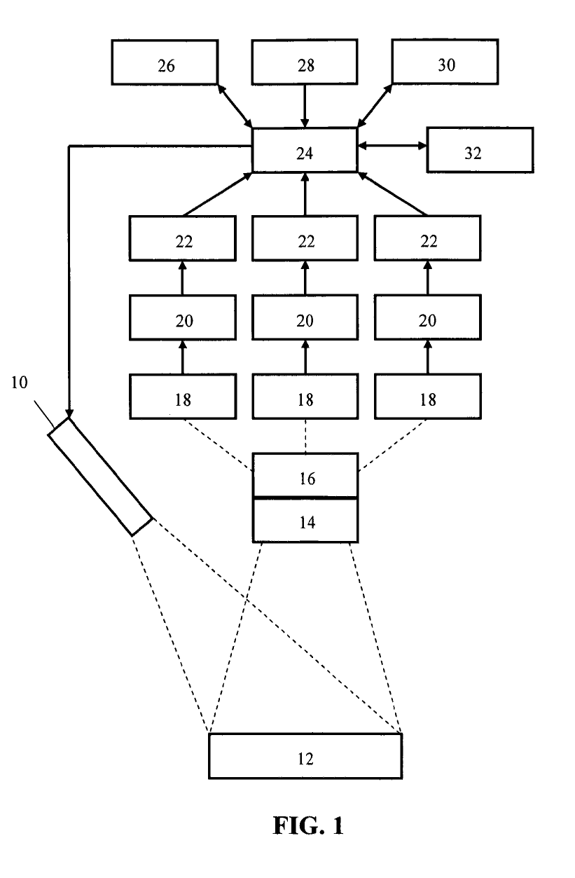

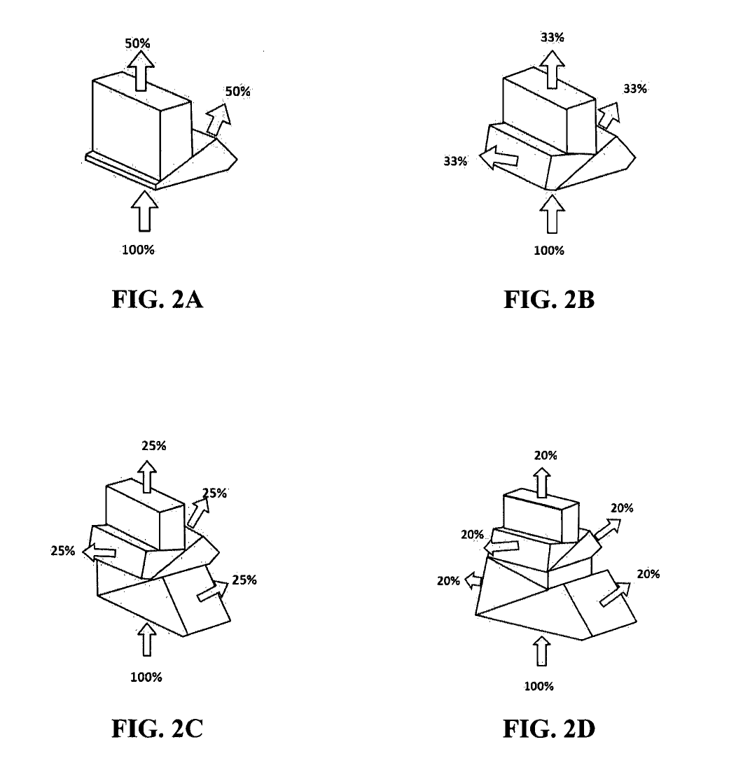

The present invention involves a system and method for real-time perfusion imaging. Referring to FIG. 1, the system includes a coherent light source 10 providing light (indicated by dashed lines) which illuminates at least a portion of an imaging subject 12. Diffusely reflected light from the imaging subject 12, referred to as spectral content, is collected by an imaging lens 14 and passes through a neutral separation prism 16. The prism 16 directs identical spectral content to each of a plurality of imaging arrays 18. FIGS. 2A-2D depict four embodiments of a separation prism 16 for directing identical spectral content to two, three, four, or five imaging arrays 18, respectively. In a preferred embodiment, the system includes three to five non-integrating CMOS imaging arrays 18 and a prism 16 capable of directing identical spectral content to that number of imaging arrays 18. Preferably, each imaging array 18 has a spatial resolution of greater than 256×256 pixels, and ideally, a resolution of at least about 750×480 pixels. Each imaging array 18 uses either an identical filter to selectively pass the light at the wavelength of the coherent light source 10 (a band pass filter) or no filter, to maintain the uniformity of the spectral content received by each imaging array 18.

A CMOS imaging array 18 includes a two-dimensional array of pixels. Each pixel transmits a current based on the intensity of incident light in a non-integrating manner, i.e., each pixel provides a distinct signal. Each imaging array 18 is in electronic communication with an analog-to-digital converter (“ADC”) 20, which converts this analog signal, i.e., the current, into a digital signal and passes that digital signal to a signal transmitting unit 22. Each of the imaging arrays 18 includes independently controllable timing, such that each of the plurality of imaging arrays 18 provides a temporally offset signal to each signal transmitting unit 22. Each signal transmitting unit 22 converts its received digital signal into image data, such as a bitmap file or other suitable image data format. Each of the signal transmitting units 22 is in electronic communication, such as, for example, via a universal serial bus (“USB”), with a central processing unit (“CPU”) 24, a processor, and transfers image data thereto.

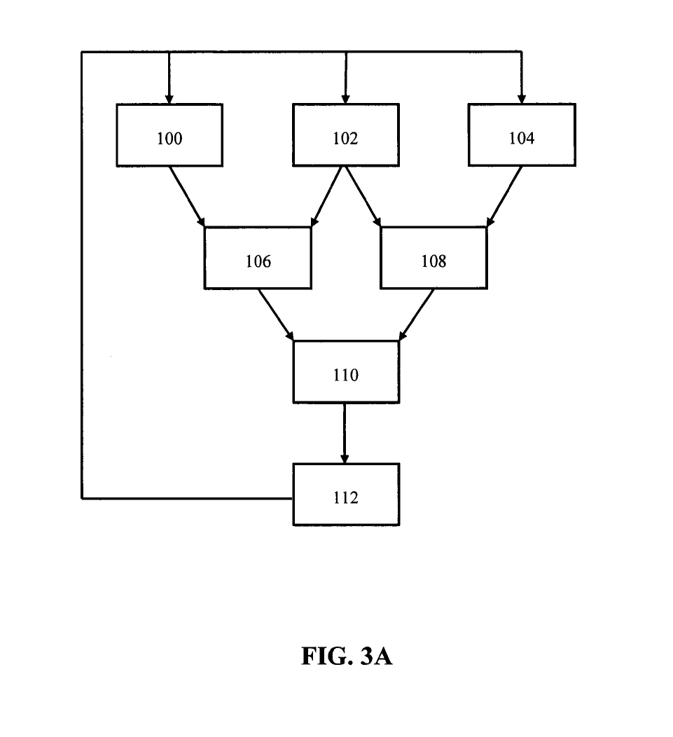

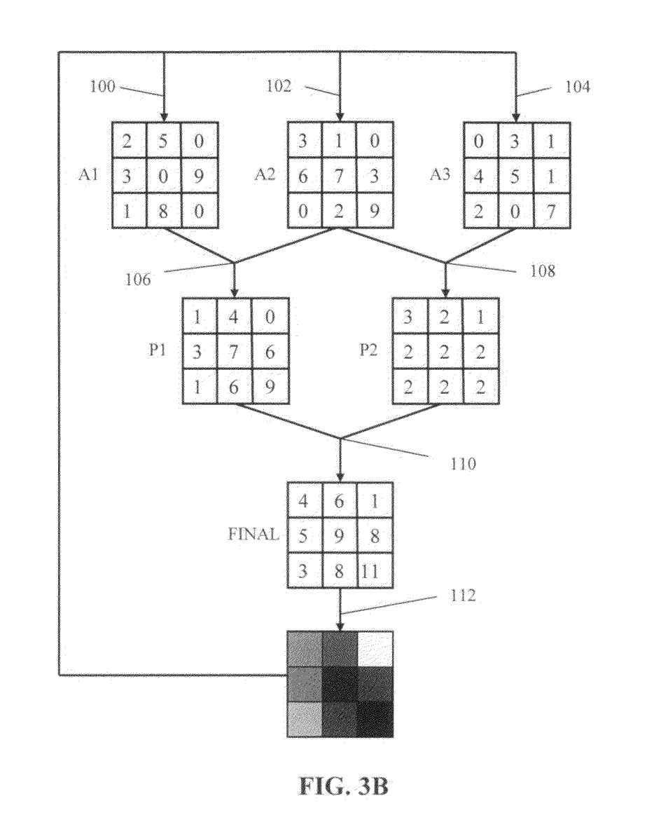

Referring now to FIGS. 3A and 3B, the central processing unit 24 generates a perfusion image from the sequentially acquired, temporally offset signals received from the plurality of imaging arrays 18. In this embodiment, the system includes three imaging arrays 18: imaging array 1 (“A1”), imaging array 2 (“A2”), and imaging array 3 (“A3”). Each of imaging array 18 includes a two dimensional pixel array defined for A1 as (A1X1,A1Y1) . . . (A1Xn,A1Yn), for A2 as (A2X1,A2Y1) . . . (A2Xn,A2Yn), and for A3 as (A3X1,A3Y1) . . . (A3Xn,A3Yn). For example, if A1 had a resolution of 750×480 pixels, its resulting image data could be interpreted as a two dimensional array of numerical values ranging from (A1X1,A1Y1) to (A1X750,A1Y480).

Each imaging array 18 includes controllable timing, such that each imaging array 18 may acquire a temporally offset image. The three imaging arrays 18 A1, A2, and A3 are timed to sequentially provide signals in a staggered fashion using a non-integrating mode. Each imaging array 18 may use a “global shutter” method of image acquisition, whereby a value for each pixel in a given array is recorded at a single time point. Alternatively, each imaging array 18 may use a “rolling shutter” method of image acquisition, whereby a value for each pixel in a given array is recorded sequentially over time. The rolling shutter method proceeds at the same rate for each imaging array 18, so the temporal offset between image acquisition at corresponding pixels on separate arrays remains constant under either method of image acquisition.

Each imaging array 18 uses an identical exposure time, the value of which may be set by the user. The exposure time is preferably set between 0.1 and 10 msec for imaging blood flow. Imaging subjects with darker skin tones require exposure times at the higher end of this range.

In step 100, A1 acquires its image and begins relaying its signal at time T1=t0. In step 102, A2 acquires its image and begins relaying its signal at time T2=t0+Δt. In step 104, A3 acquires its image and begins relaying its signal at time T3=t0+2Δt. The three arrays provide pixel to pixel correlations that are offset in time by Δt.

The CPU 24 receives signals from A1 and A2 and, in step 106, calculates a new 2D array, an absolute value array based on the pixel by pixel absolute difference between A1 and A2: |(A1X1,A1Y1)−(A2X1,A2Y1)|,|(A1X2,A1Y1)−(A2X2,A2Y1)| . . . |(A1Xn,A1Yn)−(A2Xn−A2Yn)|. This new array P1 is a record of the speckle fluctuation between T1 and T2. The CPU 24 receives a signal from A3 and, in step 108, calculates a new 2D array, P2, based on the pixel by pixel absolute difference between A2 and A3: |(A2X1,A2Y1)−(A3X1,A3Y1)|,|(A2X2,A2Y1)−(A3X2,A3Y1)| . . . |(A2Xn,A2Yn)−(A3Xn−A3Yn)|. This new array P2 is a record of the speckle fluctuation between T2 and T3. In step 110, the CPU 24 calculates a new array, an additive array based on the pixel by pixel addition of P1 and P2: (P1X1,P1Y1)+(P2X1,P2Y1), (P1X2,P1Y1)+(P2X2,P2Y1) . . . (P1Xn,P1Yn)+(P2Xn,P2Yn). This final array FINAL describes the total speckle fluctuation between T1 and T3. In step 112, the CPU 24 assigns colors to each position within the FINAL array based on the value at that location. The FINAL array is then displayed on a display 26 as a color perfusion image. The system repeatedly acquires images at a given frame rate, constantly recalculating P1, P2, and FINAL, and updating the perfusion image presented on the display 26 at a rate equal to the frame rate, which provides the user with real-time perfusion imaging. FIG. 3B depicts an example of this method of generating a perfusion image using simplified 3×3 arrays.

In an embodiment of the system including four imaging arrays 18, A1-A4, the image data in A1-A4 are sequentially acquired with a timing offset of Δt between each acquisition. Three absolute value arrays are calculated by determining the absolute difference between each pair of sequentially acquired arrays: P1 (|A1−A2|), P2 (|A2−A3|), and P3 (|A3−A4|). The three absolute value arrays are added to create the array FINAL, which describes the total speckle fluctuation between T1 and T4. The same methodology applies for embodiments of the system including five or more imaging arrays 18, A1-An. The absolute difference between each pair of sequentially acquired arrays is calculated to create absolute value arrays P1−P(n−1). Each absolute value array is then additively combined to create the array FINAL, which describes the total speckle fluctuation between T1 and Tn. Using a higher number of arrays provides increased resolution of perfusion values resulting from the increased magnitude of values in the array FINAL.

In an embodiment of the system including two imaging arrays 18, A1 and A2, a single absolute value array is calculated by determining the absolute difference between A1 and A2. This absolute value array is the array FINAL, which describes the total speckle fluctuation between T1 and T2.

Data acquired from embodiments of the system containing different numbers of imaging arrays 18 may be compared by dividing the values in each array FINAL by the number of arrays minus 1 (the number of absolute value arrays) to produce an averaged value. For example, data acquired by a five array device and data acquired by a three array device may be compared by dividing each value in the FINAL array in the five array device by four, and dividing each value in the three array device by two. However, this operation producing averaged values in the array FINAL requires floating point calculations, which are significantly more computationally intensive than the integer-only calculations used with non-averaged FINAL arrays, so the calculation and use of averaged values is not a preferred method.

The selection of the timing offset, Δt, is based on the need to resolve 20 kHz frequency components in measuring blood flow. Experimental results have indicated that the value of At should preferably be about 50 μsec. At this values of Δt, the timing offset between measurements is less than or equal to the period of the 20 kHz frequency components of blood flow. When used for purposes other than the measurement of blood flow, the timing offset may be set to any suitable value less than or equal to the maximum frequency components of the imaging subject 12.

The selection of the timing offset, Δt, is not impacted by the number of imaging arrays 18 used. Whether two imaging arrays 18 or five imaging arrays 18 are used, the appropriate value of Δt for imaging blood flow remains preferably about 50 μsec.

The system is designed to acquire images at standard imaging array frame rates: between about 30-60 frames per second (“FPS”). With three imaging arrays 18 acquiring images at a frame rate of 60 FPS, a pixel intensity fluctuation history of 180 samples (3 arrays×60 images) for each pixel is collected in only 1 second. The imaging arrays 18 are operating at their optimally designed full frame rate, so low intensity illumination is adequate and spatial resolution can be achieved by simply using imaging arrays with the desired resolution. In current perfusion imaging techniques, the total imaging time is a function of spatial resolution (number of pixels in the array). In contrast, the total imaging time in the system described herein is independent of spatial resolution and limited only by the processor speed of the CPU 24. The co-alignment and time delay between arrays allows spatially correlated pixels from the three arrays to capture the high frequency spectrum of typical blood flow.

The system includes a control set 28 for receiving input and providing output. The invention also includes a data transfer unit 30, such as, for example, a USB port, integrated wireless network adapter, Ethernet port, IEEE 1394 interface, serial port, smart card port, or other suitable means for transferring electronic data to and from the perfusion imaging apparatus. The control set 28 receives user input and allows the user to select parameters for image acquisition, such as selecting a value for the temporal offset Δt. In a preferred embodiment, the display 26 is a touch screen display capable of receiving user input, such that the control set 28 and display 26 are both embodied in a single device. The system also includes computer readable storage media 32 for retaining image data.

The system includes a coherent light source 10. In one embodiment, the coherent light source 10 is a laser emitting at less than 500 mW, and preferably about 10 mW. The coherent light source 10 emits at a specific wavelength, preferably a wavelength of at least 400 nm. Wavelengths in the visible spectrum provide the advantage that the user may see the emitted light and better orient the illumination on the imaging subject 12. Furthermore, experiments have well quantified the depth of penetration of light at 650 nm for individuals with light skin tones. In some embodiments, it may be preferable to use a coherent light source emitting in the near infrared, such as, for example, at about 850 nm. At this longer wavelength, light has improved penetration characteristics, which is necessary for imaging individuals with darker skin tones. If additional penetration is necessary, such as in an embodiment designed to scan through the clothing or wound covering of an imaging subject 12, longer wavelengths of light, such as terahertz radiation, may be used.

The performance of Doppler perfusion imaging concepts is limited by the light interference contrast in the detected optical signal. The primary practical limitation on this signal contrast is the size of the light interference pattern in relation to the size of the imaging element of the perfusion imaging system, namely, the size of the pixel in the imaging array. A typical pixel size within an imaging array 18 is a square with sides about 10 microns in length, although smaller and larger pixels are used. For maximum contrast the size of the light interference pattern should be equal to or greater than the size of the pixel. The size of the light interference pattern (speckle size) in an imaging system is defined by the equation: 2.44×wavelength×magnification×f^{#}. f^{#} is the focal length of the imaging lens 14 divided by the effective aperture diameter. Increasing the f^{#} proportionally increases the size of the interference pattern, which increases the perfusion signal contrast, which increases the resolution, accuracy and dynamic range of the perfusion imaging system. However, an increase in f^{#} generally results in a proportional decrease in light level as the aperture diameter decreases, resulting in a decrease in image intensity.

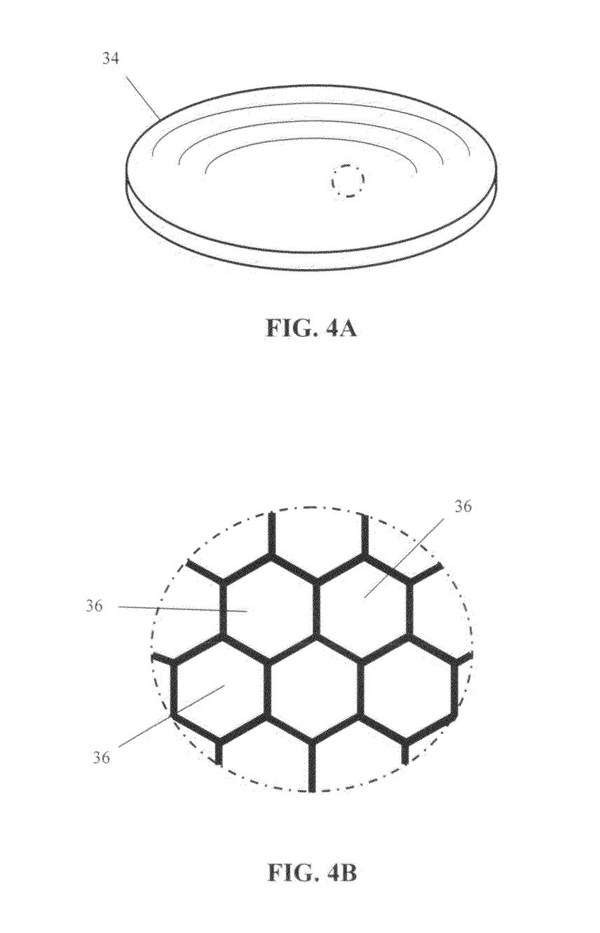

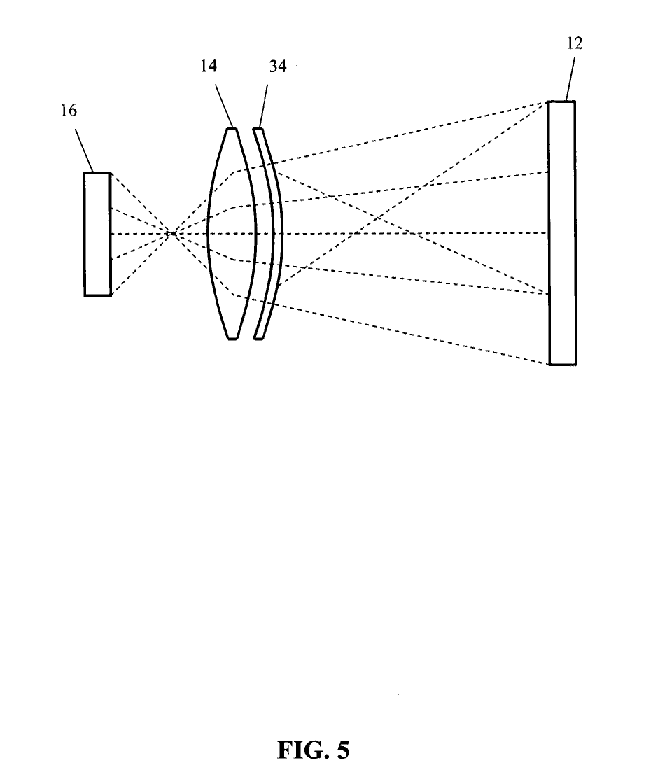

The system preferably includes a means for effectively increasing the f^{#} of the imaging system with regard to the speckle size calculation, while not proportionally decreasing the intensity of the image. As shown in FIGS. 4A, 4B, and 5, the perfusion imaging system preferably includes a microchannel array spatial filter (“MASF”) 34. The MASF 34 is constructed of optically opaque plate glass and contains a high density array of microchannels 36. The MASF 34 acts as a spatial filter, restricting the angle of light reflected off the imaging subject 12 which reaches the imaging lens 14, as depicted in FIG. 5. In one embodiment, as shown in FIG. 4B, the array of microchannels 36 on the MASF 34 are arranged in a honeycomb pattern. The microchannels may be circular or any other geometric shape in cross-section, however a hexagon shape, as shown in FIG. 4B, is preferred to maximize light throughput. As shown in FIGS. 4A and 5 the MASF 34 is preferably curved or shaped to correspond to the shape of the imaging lens 14, such that each microchannel 36 is aligned with the primary ray of the imaging system at the location of the microchannel 36, and is located adjacent to the imaging lens. The dimensions of MASF 34 equal or exceed the dimensions of the outer surface of the imaging lens 14.

The diameter and length of each microchannel 36 cooperatively determine the cone angle of light that can pass through the microchannel. Light rays approximately normal to the MASF 34 pass through the microchannels 36 and imaging lens. 14 to create an image. Higher angle rays are restricted by the MASF 32, which effectively increases the f^{#} and increases the interference pattern size (speckle size). The smaller the diameter and the greater the length of a microchannel 36, the greater the restriction of the cone angle for incoming light rays. At a microchannel diameter/length ratio of 1.0, the accepted cone angle for incoming light is about +/−45°. A decrease in the microchannel diameter/length ratio decreases the cone angle, which provides a greater increase in the effective f^{#}, but also decreases the total amount of light received by the imaging array. Using the MASF 34 reduces amount of received light, but doing so through a plurality of microchannels 36 blocks less total light than restricting a single f^{#} iris. The preferred ratio of microchannel diameter/length varies based on the pixel size and magnification of the system, with the goal being producing a speckle size approximately equal to the pixel size. A speckle size approximately equal to the pixel size maximizes perfusion image contrast while minimizing the reduction of received light.

Although the present invention is discussed in terms of diagnosis, evaluation, monitoring, and treatment of skin disorders and damage, the present invention may be used in connection with medical conditions apart from skin or for non-medical purposes. For example, the present invention may be used by a skin chemist developing topical creams, skin care products, or health or beauty aids, as it would allow quantified determination of the efficacy of the products. The present invention may also be used in connection with the sale of such products, as a salesperson could provide images of a customer's skin before and after application of a product to show the product's efficacy.

Although the present invention is discussed in terms of visualization of perfusion, it is applicable to other fields where moving and non-moving particles interact with coherent light.

The perfusion imaging system described herein may be embodied in a handheld device. In one embodiment, the perfusion imaging system is incorporated into a convergent parameter instrument, as described in a co-pending U.S. patent application for a “Convergent Parameter Instrument” filed by the inventors and incorporated herein by reference. Perfusion imaging data may be compared to or integrated with thermal imaging, near infrared imaging, high-resolution color imaging, 3D scanning, or other forms of imaging data to synergistically provide additional information to a user. In such an embodiment, elements of the system, such as, for example, the CPU 24, display 26, control set 28, data transfer unit 30, and computer readable storage media 32, may be shared with other skin parameter instruments.

The foregoing detailed description is given primarily for clearness of understanding and no unnecessary limitations are to be understood therefrom for modifications can be made by those skilled in the art upon reading this disclosure and may be made without departing from the spirit of the invention and scope of the appended claims.

What is claimed is:

1. An apparatus for imaging and measuring time-based fluctuations in a laser speckle pattern, comprising:
a plurality of coaligned imaging arrays, each with independently controllable timing;
a coherent light source arranged for illuminating at least a portion of an imaging subject;
a lens for collecting light from the imaging subject;
a prism for presenting identical spectral content from the lens to each of the plurality of imaging arrays;
a central processing unit in electronic communication with the coherent light source and each of the plurality of imaging arrays;
a control set in electronic communication with the central processing unit, the control set for controlling the coherent light source and the plurality of imaging arrays; and
a display in electronic communication with the central processing unit.

2. The apparatus of claim 1, further comprising a spatial filter arranged for restricting the cone angle of light reaching said lens.

3. The apparatus of claim 2, wherein said spatial filter includes a plurality of microchannels, each microchannel including a diameter and a length.

4. The apparatus of claim 3, wherein said plurality of microchannels are an array of microchannels arranged in a honeycomb pattern.

5. The apparatus of claim 3, wherein each of said plurality of microchannels is hexagonal in shape.

6. The apparatus of claim 3, wherein each of said plurality of imaging arrays comprises a two-dimensional array of pixels, and wherein said spatial filter includes a plurality of microchannels with a diameter/length ratio selected to produce a speckle approximately equal in size to one of said pixels.

7. The apparatus of claim 1, wherein each of said plurality of imaging arrays comprises a non-integrating two-dimensional array of pixels.

8. The apparatus of claim 1, wherein each of said plurality of imaging arrays sequentially acquires image data.

9. The apparatus of claim 8, wherein said sequentially acquired image data are separated by a user-determined temporal offset.

10. The apparatus of claim 9, wherein said temporal offset is about 50 μsec.

11. The apparatus of claim 8, wherein a perfusion image is created by calculating pixel intensity fluctuations between said sequentially acquired image data.

12. The apparatus of claim 1, wherein each of said plurality of imaging arrays acquires image data at a frame rate between about 30-60 frames per second.

13. The apparatus of claim 1, wherein said coherent light source emits at a wavelength of at least 400 nm and at a power of less than 500 mW.

14. The apparatus of claim 13, wherein said coherent light source emits at a wavelength between about 630-850 nm and at a power of about 10 mW.

15. The apparatus of claim 13, each of said plurality of imaging arrays further comprising a band pass filter arranged to selectively pass light at about the wavelength of said coherent light source.

16. The apparatus of claim 1, said apparatus being incorporated into a convergent parameter instrument.

17. The apparatus of claim 1, wherein said independently controllable timing of each of said plurality of imaging arrays may be controlled by a user.

18. The apparatus of claim 1, wherein said plurality of imaging arrays is one of three, four, and five imaging arrays.

19. The apparatus of claim 1, wherein said display is a touch screen display.

20. The apparatus of claim 1, further comprising a data transfer unit in electronic communication with said central processing unit.

21. The apparatus of claim 1, further comprising computer readable storage media in electronic communication with said central processing unit.

22. A method for producing a perfusion image comprising the steps of:
a. illuminating at least a portion of an imaging subject using a coherent light source;
b. collecting spectral content from said imaging subject;
c. directing the spectral content to each of a plurality of imaging arrays;
d. sequentially acquiring an image with each of said plurality of imaging arrays;
e. processing said sequentially acquired images to generate a single perfusion image; and
f. displaying the perfusion image on a display.

23. The method of claim 22, wherein the sequentially acquired images of step d are separated by a user-determined temporal offset.

24. The method of claim 22, wherein each of said sequentially acquired images is represented as a two dimensional array of numerical values.

25. The method of claim 24, wherein the processing of said sequentially acquired images in step e comprises:
a. calculating an absolute value array for each pair of sequentially acquired images, whereby each position in the absolute value array is the absolute value of the difference in numerical values at corresponding positions in the temporally adjacent pair of sequentially acquired images;
b. determining if only a single absolute value array was calculated in step a:
1. if so, defining the single absolute value array as a final array and proceeding to step c;
2. if not, calculating an additive array, whereby each position in the additive array is the sum of numerical values at corresponding positions in each of the absolute value arrays, defining the additive array as a final array, and proceeding to step c;
c. assigning a color to each position within the final array based on the numerical value at that position to generate a perfusion image.

26. The method of claim 25 performed by a central processing unit in electronic communication with said coherent light source and said plurality of imaging arrays.

27. The method of claim 22, whereby steps a-f are repeated at a rate between about 30-60 Hz for displaying real-time perfusion imaging.

28. The method of claim 22, wherein said imaging subject is an individual's skin.
